(19) United States
(12) Patent Application Publication
(10) Pub. No.: US 2018/0267286 A1
(43) Pub. Date: Sep. 20, 2018

(54) Observation Device with Optical Compensation

(71) Applicant: ECOLE POLYTECHNIQUE FEDERALE DE LAUSANNE (EPFL), Lausanne (CH)

(72) Inventors: David Vincent Bonzon, Le Mont-Pèlerin (CH); Steve Béguin, Hawthorn East (AU); David Forchelet, Coffrane (CH); Philippe Renaud, Préverenges (CH); Yann Barrandon, Echandens (CH); Georges Henri Muller, Lausanne (CH)

(21) Appl. No.: 15/544,520

(22) PCT Filed: Jan. 21, 2016
(86) PCT No.: PCT/IB2016/050295

§ 371 Date: Jul. 19, 2017

(30) Foreign Application Priority Data
Jan. 27, 2015 (IB) PCT/IB2015/050624

(51) Int. Cl. G02B 21/34; C12M 1/32; C12M 1/34; G02B 21/06

(52) U.S. Cl. CPC G02B 21/34; C12M 23/12; B01L 2300/0681; G02B 21/06; C12M 41/36

(57) ABSTRACT

The invention concerns an observation device such as cell culture wells comprising an optical element such as a lens or a filter, or even combinations thereof, for compensating an optical effect induced on a sample contained in the observation device and illuminated by a light beam traversing a meniscus. The interaction of a light beam with a meniscus interposed between said light beam and a sample to be visualized, alters the image readout of the sample so that the image thereof results negatively affected. By aligning the optical axis of the optical element with that of the meniscus, an optical effect such as non-uniform light distribution of illumination of the sample can be conveniently compensated. The invention further discloses the optical elements, characterising the observation device, per se.

### TECHNICAL FIELD

The invention generally pertains to the field of optics and more precisely to the observation of illuminated objects within a fluid.

### BACKGROUND ART

The ability to image cultured cells is crucial for the understanding and control of biological processes. Imaging of cells benefits many applications including biotherapeutics, drug discovery, cancer research and regenerative medicine. Moreover, high-quality images are crucial to implement high throughput automated image analysis. Current limitations to achieve high-quality images impose to pre-treat the acquired images, a process that is not perfect and which may present a risk of inducing errors as well as creating artifacts.

Multiwell (or microtiter or microwell) plates have become an indispensable tool for the growing field of live-cell studies. Wells can be circular or square, have straight, stepped, or curved walls and a flat surface where cells can adhere and can be cultured. A multiwell plate typically has 6, 24, 96, 384 or even 1536 sample wells arranged in a 2:3 rectangular matrix. Among them, the 96-well format is very convenient for low to middle throughput experiments. In fact, they still offer a decent parallelization in the experiments and can still be handled manually without the need for expensive robots. From the view of industrialization, the manufacture of multiwell plates is rendered possible thanks to well-described plastic injection molding and assembly processes (US2005/0047971A1).

In order to culture cells, multiwell plates are filled with culture media or buffer solutions. Depending on the surface energy of the materials of the wells, the wetting of the inner surfaces of said well is affected and a meniscus forms at the air-liquid interface due to capillary forces.

The multiwell plates are still highly limiting when it comes to cell microscopy.

Originally, the multiwell plates were designed for analytical research and clinical diagnostic laboratory testing for which imaging was not considered.

To perform cell microscopy, the multiwell plate is placed under a microscope. This meniscus being in the illumination path acts as a lens and degrades the imaging readout, for example in terms of homogeneity of illumination phase and intensity of the sample. As illustrated in FIG. 1, the images taken in a 96-well plate suffer from illumination issues resulting in poor image quality.

Several solutions have been proposed to solve the meniscus-related issues on imaging of multiwell plates. First, a method was established to reduce meniscus curvature by specific wall surface treatments (US 2010/0047845A1). This results into an inhomogenetity of surface energies of the well. Consequently, it may modify the wettability of the well with the risk of introducing air bubbles that impacts the cell culture and imaging. In a second invention (U.S. Pat. No. 8,703,072), cell culture vessels were designed with surface features overlying the interior surface of the well. The features aim to alter the contact angle between the liquid and the wall of the well, thus reducing the meniscus. However, the suggested wall geometry makes the plates challenging to manufacture with standard injection molding processes. In a third patent, the meniscus is eliminated with the insertion of a plug into the well (U.S. Pat. No. 6,074,614). In practice, such plugs are arranged on a plate cover and must be aligned to the multiwell plates. When the cover is removed from the plate, the plugs may carry droplets of liquid, which may lead to unwanted (cross-)contaminations between the wells. In conclusion, existing solutions are inappropriate to correct the meniscus optical effect mostly because they are invasive methods. Thus, there is a need for alternative solutions compensating the meniscus adverse optical effects to improve imaging of samples in multiwell plates without altering standard laboratory practices.

### SUMMARY OF INVENTION

It is an object of the present invention to provide for an observation device characterized in that it comprises an accessory construed for compensating adverse optical effects that can be generated when a light beam interacts with a meniscus created on the interface of two fluids, such as for instance a meniscus on a liquid-air interface. The invention is particularly useful in imaging settings, where an operator is intended to analyse an object or a sample, illuminated by a light beam, via an imaging system, wherein the image of such an object is distorted or otherwise altered by the optical effect induced by a meniscus interposed between the light source and the sample to be analysed. Upon the interaction of the light beam with the meniscus, said light beam can be modulated, deviated or otherwise modified in several ways, depending on many factors such as the difference in refractive index of the fluids, the optical path of the light through the fluids, the absorption coefficient of the fluids, the curvature of the meniscus and so forth. As a consequence, the light beam can be totally or partially refracted, diffracted, reflected or diffused so that the quality of the image of the illuminated sample results negatively affected, for example in terms of homogeneity of illumination intensity and phase distribution on the imaged sample. The present inventors came up with a simple an elegant solution to tackle and overcome such a problem, as described hereinafter and in the appended claims.

Accordingly, in one embodiment, the invention features an observation device comprising a container for a fluid, having a bottom and an openable upper end, and which is dimensioned in a way as to shape a meniscus on the interface between the contained fluid and a second fluid, wherein the bottom of the container is adapted to accommodate a sample visualizable with an imaging system once illuminated by a light beam, said device being characterized in that it furthermore comprises an optical element aligned with the optical axis of the meniscus illuminated by a light beam and adapted to compensate an optical effect induced by said meniscus on the sample.

In a preferred embodiment, the fluid contained in the container is a liquid. In a further preferred embodiment, the observation device of the invention is a well plate, a petri dish or a multiwell plate for cell culture.

In a preferred embodiment, the optical effect compensated by the optical element is the distribution of the illumination intensity, phase, wavelength and/or polarization on the imaged sample.

In one embodiment, the optical effect compensated by the optical element is a monochromatic or a chromatic aberration.

In a preferred embodiment, the optical element of the observation device comprises at least one optical filter, at least one lens or combinations thereof. In a further preferred embodiment, the at least one optical filter is a spatial gradient filter. In a more preferred embodiment, the spatial gradient filter is a radial gradient filter or a light intensity filter.

In a further preferred embodiment, the optical element is placed in, on or under the openable upper end and/or the bottom of the observation device.

In a further aspect, the invention features an optical element for an observation device as previously defined.

### BRIEF DESCRIPTION OF DRAWINGS

FIG. 1 shows cells cultured on a 96-well plate and imaged with a 10× phase contrast microscope. In order to screen all the well, a total of 30 images should be acquired and stitched together.

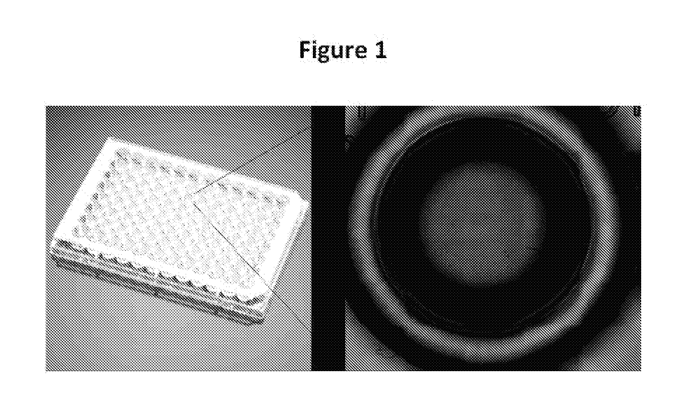

FIG. 2 shows an embodiment of the working principle of the observation device of the present invention comprising an optical element. Microscopy in conventional plates gives low quality imaging. The refraction, due to the meniscus, disarranges the correct alignment of the light path, resulting in an inhomogeneous illumination of the imaged area. The optical element compensates for the adverse optical effect by adapting the illumination intensity distribution in its filter-like embodiment, or the optical path of a light beam in its lens-like embodiment, in order to compensate for the illumation inhomogeneity, so that the imaging area is homogeneously illuminated. Optimal homogeneous illumation intensity leads to a much better image acquisition. Cells can be imaged with high quality with standard microscopy techniques such as phase contrast or bright field microscopy.

FIG. 3 shows the observation device of the invention (in this case, a multi-well plate) comprising an optical element arranged on the top thereof. Filter version embodiment (left) and lens version embodiment (right) are shown.

FIG. 4 shows phase contrast microscopy in a conventional 96-well plate. The use of the compensative optical element of the invention (right panel) shows a more homogeneous distribution illumination intensity than without using it (left panel).

FIG. 5 shows a further setting of the optical element according to the present inventive concept. Several optical elements can be combined in many different arrangements, such as for instance stacked one over the other, and aligned anywhere along the optical path of the light beam traversing the fluid container.

### DESCRIPTION OF EMBODIMENTS

The present disclosure may be more readily understood by reference to the following detailed description presented in connection with the accompanying drawing figures, which form a part of this disclosure. It is to be understood that this disclosure is not limited to the specific conditions or parameters described and/or shown herein, and that the terminology used herein is for the purpose of describing particular embodiments by way of example only and is not intended to be limiting of the claimed disclosure.

As used herein and in the appended claims, the singular forms “a”, “an” and “the” include plural referents unless the context clearly dictates otherwise.

Thus, for example, reference to “an optical element” includes a plurality of such elements and reference to “an optical effect” includes reference to one or more of such effects, and so forth.

Also, the use of “or” means “and/or” unless stated otherwise. Similarly, “comprise,” “comprises,” “comprising” “include,” “includes,” and “including” are interchangeable and not intended to be limiting. It is to be further understood that where descriptions of various embodiments use the term “comprising”, those skilled in the art would understand that in some specific instances, an embodiment can be alternatively described using language “consisting essentially of” or “consisting of.”

As used herein, an “observation device” is any device or article of manufacture in general that permits to accommodate a sample on and/or within it and to suitably adapt said sample in order to be visualized, preferably with an imaging system. The observation device according to the present invention comprises at least one container having a bottom, preferably a flat bottom, and an open or preferably an openable upper end.

The openable upper end can be for instance a lid or a microscope slide, as well as any other element suitable to close the container. The container can have any volume and three-dimensional shape, such as for instance a cylindrical or frusto-conical shape, and is preferably made of a transparent or translucent material such as glass or plastic materials such as for instance polyethylene, polystyrene, polypropylene, polycarbonate and so forth. As per its definition, a container of the observation device can contain both a sample to be visualized and a fluid, and is dimensioned in such a way as to permit the creation of a meniscus on the interface between the contained fluid and a second fluid. In a preferred aspect according to the invention, the observation device is a well plate, a petri dish or a multiwell plate for cell culture.

In the frame of the present invention, the term “optical element” refers to any accessory, device or article of manufacture in general that, when interacting with a light beam produced by a light source, acts by modifying at least one property of said light beam such as intensity, phase, propagation direction, frequency, wavelength or polarisation. The term “light” refers herein to visible light, infrared (IR) light, ultraviolet (UV) light, coherent or non-coherent light and so forth, although in a preferred embodiment of the invention the light is visible light, i.e. light having a wavelength in the range of 400 nanometres (nm) to 700 nanometres. A compensative optical element according to the present invention comprises or consists of a lens, a polarizer, a diffraction grating, a prism, a reflector, a filter, a mirror or any combination thereof.

In a preferred embodiment, the optical element according to the present invention comprises or consists of a lens. A “lens” is a transmissive optical device which affects the focusing of a light beam through refraction, i.e. the phenomenon that occurs when waves travel from a medium with a given refractive index to a medium with another at an oblique angle, causing a change in the direction of propagation of the waves as well as a phase shift.

A simple lens consists of a single piece of material, while a compound lens consists of several simple lenses, usually along a common axis. Lenses are usually made from transparent materials, ground and polished to a desired shape, but different material can be used for producing a lens according to the present invention, such as for instance hydrogels, oils, crystals such as quartz, glass based material such as crown borosilacte, calcium fluoride or organic materials such as polycarbonate, thiocarbamates, polymethylmetacrylates, polysterene. Lenses are classified by the curvature of the two optical surfaces. A lens is biconvex (or double convex, or just convex) if both surfaces are convex. If both surfaces have the same radius of curvature, the lens is equiconvex. A lens with two concave surfaces is biconcave (or just concave). If one of the surfaces is flat, the lens is plano-convex or plano-concave depending on the curvature of the other surface. A lens with one convex and one concave side is convex-concave.

According to the present invention, particularly suitable lenses for obtaining the desired effect (i.e., the compensation or correction of the optical effect on a sample illuminated by a light source due to the interaction of a light beam with a fluid-fluid interface meniscus between the light source and the object) are, but not limited to, biconvex or plano-convex lenses and Fresnel lenses.

In another preferred embodiment, the compensative optical element according to the present invention comprises or consists of at least one optical filter. For “optical filters” are herein meant devices that selectively transmit light of different wavelengths and/or in a particular range of wavelengths while blocking totally or partially the remainder. Optical filters can be used to attenuate light intensity by transmitting, blocking or reflecting specific wavelengths. Filters mostly belong to one of two categories. “Absorptive filters” are usually made from a translucent material to which various inorganic or organic compounds have been added. These compounds block totally or partially some wavelengths of light while transmitting others. Alternately, “dichroic filters” (also called “reflective” or “thin film” or “interference” filters) can be made by coating a glass or any other suitable substrate with a series of optical coatings. Dichroic filters are used to selectively pass light of a small range of colours and usually reflect totally or partially the unwanted portion of the light while transmitting the remainder.

Many different shapes can be envisaged for the filters according to the present invention, and many different materials can be used to make such filters like for instance crystals such as quartz, glass, polymers such as polystyrene, polymethylmetacrylate, polycarbonate, polyethylene terephthalate, cellulose with absorbing ink, dye, particles, metallic thin film or any other suitable material, as long as filters remain able to totally or partially block light in particular range of wavelength. A particularly suitable optical filter that can be used in the frame of the present invention is a spatial gradient filter. A “spatial gradient filter” is an absorption or even a dichroic filter whereby the capacity of light absorption varies in a spatial fashion, as example radially in a plane parallel to the one of the sample being imaged.

A spatial gradient filter is particularly appropriate when a inhomogeneity of the illumination of the sample to be visualized, once placed in the conditions as described above and in the appended claims, is intended to be compensated. In particular, when the intensity of the illumination is inhomogeneous on the sample to be analysed with an imaging system, due to the meniscus on the fluid-fluid interface and its resulting convergent or divergent lens effect, a spatial gradient filter permits to compensate for the inhomogeneity of illumination intensity distribution thanks to, for example, its shading properties on visible light.

According to the inventive concept of the invention, the optical element characterising the observation device should be aligned with the optical axis of a meniscus created on a fluid-fluid interface, said meniscus being interposed between a sample and a light source. In the frame of the present invention, the terms “aligned”, “aligning” or even “alignment” mean that the optical axis of the optical element shall be superimposed to the optical axis of the meniscus. It will be apparent for a person skilled in the relevant art that an “optical axis” is the line where a light beam travels an optical system or an optical element without experiencing any angular change while crossing said system or element, wherein the “optical system” in the present case is represented of at least one optical element and the meniscus at stake.

The optical element has a compensative effect on at least one adverse optical effect (created by the meniscus when interacting with a light beam traversing it) on the illumination of the sample to be visualized. For “adverse optical effect” is herein meant any alteration or modification of the image of the sampled object due to the change of at least one property of a light beam once interacting with a fluid-fluid interface meniscus. An adverse optical effect can be for instance the opacity, blurring or shading of all or part of the imaged sample, due e.g. to an inhomogeneous distribution of the illumination intensity or phase on the sample to be visualized. Additionally or alternatively, an adverse optical effect can be an optical aberration, such as a chromatic or a monochromatic aberration. Many kind of optical aberrations are known in the art, and they are generally defined as a departure of the performance of an optical system from theoretical predictions or a mathematical model. Aberrations fall into two classes: monochromatic and chromatic. Monochromatic aberrations are caused by the geometry of the lens or mirror and occur both when light is reflected and when it is refracted. They appear even when using monochromatic light, hence the name. Chromatic aberrations are caused by dispersion, i.e. the variation of a lens's refractive index with wavelength, and they do not appear when monochromatic light is used. Examples of optical aberrations are piston, tilt, defocus, spherical aberration, coma, astigmatism, field curvature or image distortion.

According to the invention, the above-described adverse optical effect is caused by the lens effect of the meniscus on the light beam with which it interacts. For “meniscus” is herein meant the curve in the upper surface of a fluid in a container or another object containing it, caused by capillary forces on the walls of said container. It can be either convex or concave, depending on the fluid and the surface. A convex meniscus occurs when the particles in the fluid have a stronger attraction to each other (cohesion) than to the material of the container (adhesion). Convex menisci occur, for example, between mercury and glass in barometers and thermometers.

Conversely, a concave meniscus occurs when the particles of the fluid are more strongly attracted to the container than to each other, causing the fluid to climb the walls of the container, as occurs for instance between water and glass. Menisci are a manifestation of capillary action, by which surface adhesion pulls a fluid up to form a concave meniscus or internal cohesion pulls the fluid down to form a convex meniscus. Depending on such parameters, the meniscus will behave, once traversed by a beam of light, as a concave or a convex lens, thus creating the conditions for triggering an adverse optical effect on a sampled object. However, a meniscus can be even artificially created on the boundaries of two different fluids by for instance a curve transparent, possibly flexible membrane dividing the two fluids.

As said, the meniscus is created on a fluid-fluid interface. As used herein, a “fluid” is a substance that continually deforms (flows) under an applied shear stress. Fluids are a subset of the phases of matter and include liquids, gases, plasmas and plastic solids. They display properties such as not resisting deformation, or resisting it only lightly and the ability to flow (also described as the ability to take on the shape of the container).

In a preferred embodiment of the invention, at least one fluid at the meniscus' interface comprises a liquid such as e.g. water, aqueous solutions, non-polar (e.g. oil) solutions and the like. An “aqueous solution” is a solution in which the solvent is substantially made of water. In the frame of the present disclosure, the term “aqueous” means pertaining to, related to, similar to, or dissolved in water. The expression aqueous solution in the frame of the present disclosure also includes highly concentrated and/or viscous solutions such as for instance gels or hydrogels. As used herein, the term “gel” refers to a jelly-like material composed of a colloidal network or polymer network that is expanded throughout its whole volume by a fluid.

A gel is a three-dimensional network that spans the volume of a liquid medium and ensnares it through surface tension effects. The internal network structure may result from physical bonds (physical gels) or chemical bonds (chemical gels). “Hydrogels” are gels in which the swelling agent is water. A hydrogel is a macromolecular polymer gel constructed of a network of crosslinked polymer chains. It is synthesized from hydrophilic monomers, sometimes found as a colloidal gel in which water is the dispersion medium. Hydrogels are highly absorbent (they can contain over 90% water) natural or synthetic polymeric networks. As a result of their characteristics, hydrogels develop typical firm yet elastic mechanical properties. Some examples of hydrogels include, but are not limited to, gelatin, collagen, agar, chitosan, or amelogenin.

In a further preferred embodiment, the liquid is an aqueous solution such as those used in laboratory and research settings comprising but not limited to culture media, buffer solutions, paraformaldehyde and so forth.

As said, the adverse optical effect triggered by the interaction of a light beam with a meniscus alters the image readout of a sample, comprised in the observation device, to be visualized/analysed. Such a sample, in the frame of the present disclosure, is usually visualized by means of an imaging system. As used herein, an “imaging system” is an optical instrument that either processes light waves to enhance an image for viewing or analyzes light waves to determine one of a number of characteristic properties.

Usually, imaging systems are optical tools used to aid and/or enhance vision by forming an image that is a different size or at a different position from the sample or object; however, in its simplest embodiment, even the eye can be considered a suitable imaging system, depending on the operators' needs. In certain aspects according to the present disclosure, an imaging system also permits to record and/or analyse data, as well as the acquisition thereof. Imaging systems are usually coupled with a light source used to illuminate a sample, said light source being either internal (that is, a light source incorporated within the system) or external (i.e., coming from outside of the system, such as for instance sunlight). A non-comprehensive list of suitable imaging systems according to the present invention includes magnifiers, microscopes, cameras and projectors. In a preferred embodiment according to the present invention, the imaging system is a microscope such as for instance simple or compound microscopes, inverted microscopes, bright-field or dark-field microscopes, phase contrast microscopes, polarizing microscopes, fluorescence microscopes, confocal microscopes and so forth.

### EXAMPLE

In order to accelerate development of the field of biological and biomedical research, there is a need for well plates that:
accommodate with routine microscopy techniques to give high-quality images;
fit to the standard format of culture plates to be non-disruptive with protocols and
is low cost and user-friendly to be routinely used by scientists.

The following example describes a simple optical element complement, which can be coupled or embedded in a final product, and is adaptable in principle to any well plate such as for instance 96- or 384-well plates to improve imaging readouts.

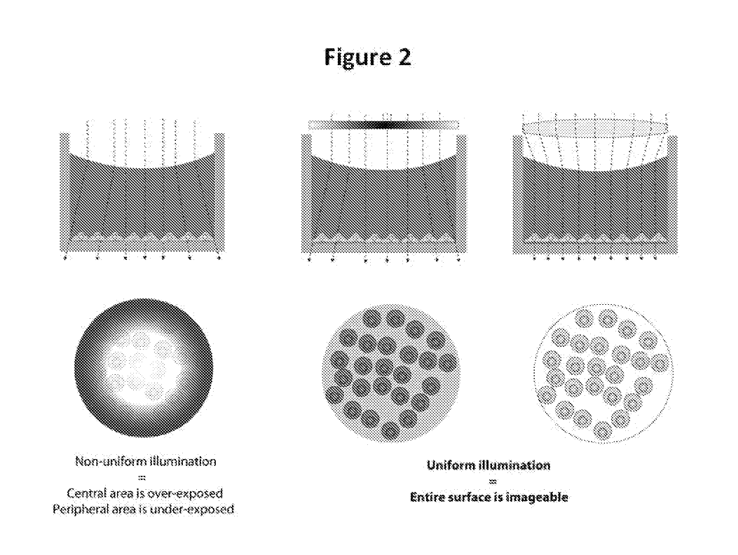

The embodiment herewith described discloses an observation device, in particular a multiwell plate, comprising an optical element used to correct the non-uniform illumination of wells, caused by the meniscus of the culturing media (or any liquid) and its resulting divergent lens effect. The result of the light inhomogeneity is the presence of over- or under-exposed areas when images are visualized through an imaging system, acquired with a camera or even seen by eye (FIG. 2, left panel).

The compensative optical element, described below as a spatial progressive (or gradient) light filter that produces a shadow, or as a lens that modifies the optical path of a light beam generated by a light source, is able to compensate for the inhomogeneity of illumination intensity distribution on the sample (FIG. 2, middle and right panels). The compensative optical element can be put anywhere on the optical path, e.g. on the lid or on the bottom of the plate, and it is therefore conveniently adaptable for most microscope techniques based on light, such as bright field, phase contrast, fluorescence, trans-illumination, reflection and so forth.

For what concerns an optical filter, it may be printed directly onto a multi-well plate, either on the lid or the bottom thereof, to be fully disposable in order to avoid sterility/contamination problems. In such a scenario, a well/multiwell plate integrates in it the filtering optical element forming the core of the present inventive concept. Additionally or alternatively, the filtering optical element can be integrated directly in the material of the lid or bottom of the container during manufacturing. Additionally or alternatively, the filtering optical element can be produced on any other suitable support designed to the standard dimensions of the wells of culture plates. In such a way, a filter element can be adapted to any kind of well and well plates for cell microscopy already on the market. Moreover, even if thought for being disposable, the corrective element can be reused many times by adapting it to more than one well/multiwell. Alignment of the corrective element with the optical axis of the culture medium's (or any other liquid) meniscus can be assured in many different ways; for instance, the optical element can be conveniently produced in form of a sticker (that represents the most preferred embodiment of the optical element comprising an optical filter), or grafted on the lid and/or the bottom of the well, so that the correct alignment is guaranteed without impairing the possibility to detach the optical element for further use.

The same concepts described above can be applied to a compensative optical element comprising one or more lenses. Said lenses can be directly produced on the lid and/or on the bottom of the well/multiwell (that is, under the flat surface of wells in order not to impair the possibility of e.g. homogeneous culture of adherent cells), so to obtain a well/multiwell integrating the optical elements in it, or they can be otherwise placed anywhere along the optical path of the light illuminating the sample in the wells in a later time, when needed. Of course, the lenses of the compensative optical element must be aligned with the meniscus of the liquid contained in the well in order to obtain the best possible result and compensate for adverse optical effects. Even in this case, the lenses can be be produced on any suitable support designed to the standard dimensions of the wells of culture plates, and can be easily attached/detached.

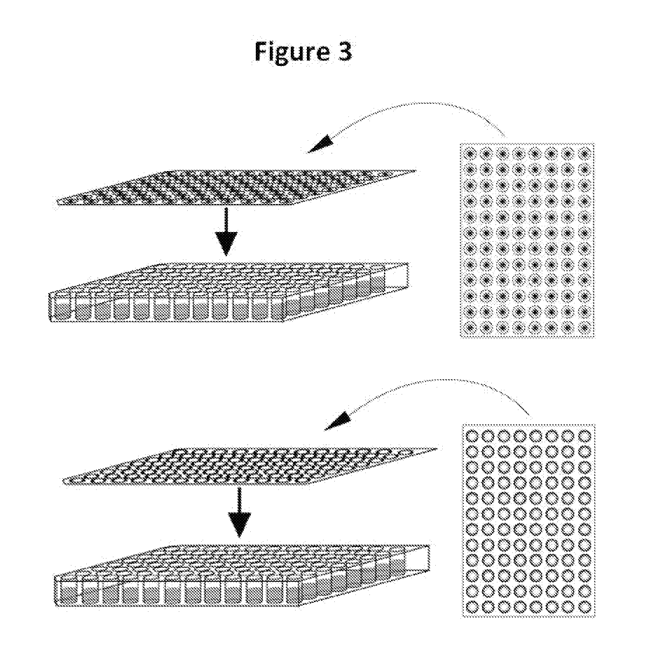

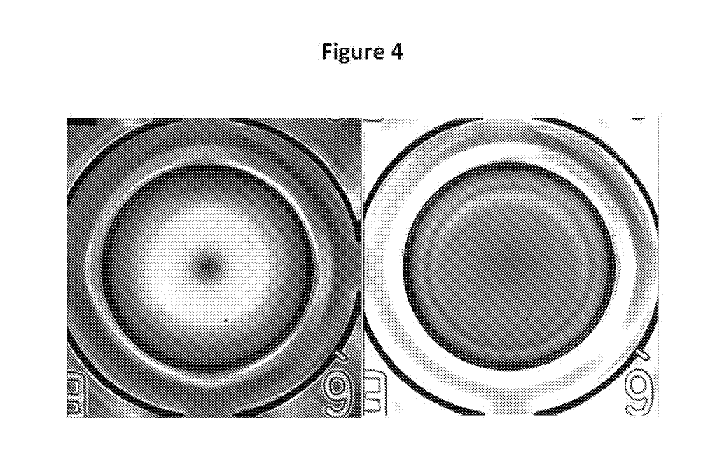

FIG. 3 shows the elements of the invention and their integration on a multi-well plate used for cell microscopy. A multi-well plate designed to the standard format of the industry is implemented with the accessory in the form of a transparent membrane with optical filters printed on it or corrective lenses produced on it. FIG. 4 shows phase contrast microscopy in a conventional 96-well plate. The use of the optical element of the invention (right panel) shows a more uniform light intensity than without using it.

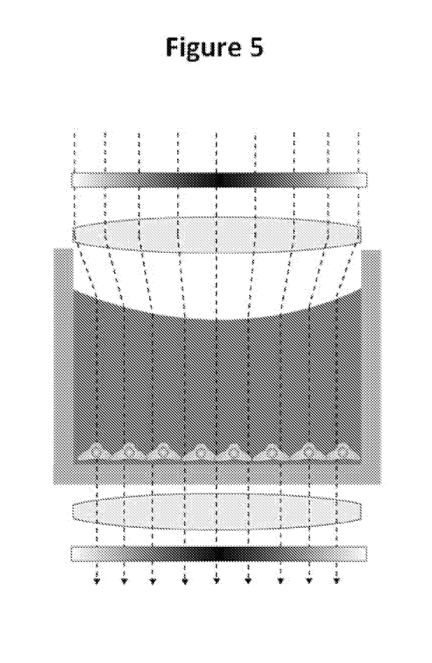

FIG. 5 shows a further setting of the optical element according to the present inventive concept. Several optical elements can be combined in many different arrangements, such as for instance stacked one over the other, and aligned anywhere along the optical path of the light beam traversing the medium-containing well. However, an optical element of the invention can even be integrated on or inside a further optical element, for instance during the manufacturing process. One example could be represented by an absorption filter created inside a lens during e.g. the crystallization step of this latter.

This approach can be particularly useful when the optical element is supposed to compensate for more than one adverse optical effect, or when further properties of the sampled object need to be analysed. In this context, for example, a lens can be combined in the optical element with a dichroic filter in order to select for a specific wavelength, while ameliorating the homogeneity of illumination of the sample. In this way, the analysis of e.g. fluorescent samples in a single cell-setting experiment could be boosted and accelerated with a simple and tailored tool.

Thus, the compensative optical elements of the invention fully integrates into laboratory protocols and equipment and it may be manufactured in such a way as to allow it to operate universally with multiwell plates or petri dishes produced by any number of manufacturers.

The most promising application fits in the production of biotherapeutics both at academic and industrial level. In biotech, cell lines must be derived from single cells to improve yield and safety of therapeutics (monoclonality).

Typically, single-cells are dispensed into multiwell plates where it is not possible to perform high quality imaging. Thus, monoclonality cannot be confirmed. By providing a visual control of the monoclonality, the disclosed embodiment of the invention drastically speeds up cell line development and consequently reduces time-to-clinic for therapeutics. As an add-on for existing multi-well plates, the optical element according to the present disclosure is an accessory that can give very high quality bright field and phase contrast images, which is particularly important for the imaging of a few numbers of precious cells, down to the single-cell level.

1-11. (canceled)

12. An observation device comprising:
a container having a bottom and an open upper end, the container dimensioned to shape a meniscus between an interface of a first fluid arranged in the container and a second fluid, the bottom of the container configured to accommodate a sample in the first fluid, the sample being viewable with an imaging system when illuminated by a light beam; and
an optical element aligned with an optical axis of the meniscus illuminated by the light beam, the optical element configured to compensate an optical effect caused by the meniscus when imaging the sample.

13. The observation device of claim 12, wherein the first fluid in the container is a liquid, and the second fluid is air.

14. The observation device of claim 12, wherein the observation device is at least one of a well plate, a petri dish, and a multi-well plate for cell culture.

15. The observation device of claim 12, wherein the optical effect includes at least one of a distribution of the illumination intensity of the light beam, phase of the light beam, wavelength of the light beam, and polarization of the light beam.

16. The observation device of claim 12, wherein the optical effect includes at least one of a monochromatic aberration and a chromatic aberration.

17. The observation device of claim 12, wherein the optical element includes at least one of an optical filter and lens.

18. The observation device of claim 17, wherein the optical filter includes a spatial gradient filter.

19. The observation device of claim 18, wherein the spatial gradient filter includes a radial gradient filter.

20. The observation device of claim 18, wherein the spatial gradient filter includes a light intensity filter.

21. The observation device of claim 19, wherein the radial gradient filter includes a light intensity filter.

22. The observation device of claim 12, wherein the optical element is at least one of arranged inside the container, arranged below the bottom of the container, and arranged above the open upper end of the container.

23. The observation device of claim 12, wherein the open upper end includes at least one of a lid and a microscope slide, configured to close the container.

24. An optical element for an observation device, the observation device comprising:
a container having a bottom and an open upper end, the container dimensioned to shape a meniscus between an interface of a first fluid arranged in the container and a second fluid, the bottom of the container configured to accommodate a sample in the first fluid, the sample being viewable with an imaging system when illuminated by a light beam; and
an optical element aligned with an optical axis of the meniscus illuminated by the light beam, the optical element configured to compensate an optical effect caused by the meniscus when imaging the sample.
